(19) United States
(12) Patent Application Publication
(10) Pub. No.: US 2026/0191548 A1
(43) Pub. Date: Jul. 9, 2026

(54) THROMBUS RETRIEVER INTRAVASCULAR DEVICE

(71) Applicant: SCIENTIA VASCULAR, INC., West Valley City, UT (US)

(72) Inventor: Edward J. SNYDER, Park City, UT (US)

(21) Appl. No.: 19/130,637

(22) PCT Filed: Dec. 21, 2023
(86) PCT No.: PCT/US2023/085342

§ 371 Date: May 16, 2025

Related U.S. Application Data
(60) Provisional application No. 63/434,427, filed on Dec. 21, 2022.

(51) Int. Cl. A61B 17/221; A61B 90/00

(52) U.S. Cl. CPC A61B 17/221; A61B 90/39; A61B 2090/3966

(57) ABSTRACT

The present disclosure relates to an intravascular device for thrombus removal. The device includes a core and a tube structure coupled to the distal section of the core. The tube structure includes a first, distal section and a second, proximal section. The device further includes a wire mesh located in a gapped region between the first section and the second section of the tube structure. The core is coupled to the first section of the tube structure but is free to move longitudinally relative to the second section of the tube structure. Application of a pulling force to the core can therefore selectively contract the wire mesh and cause it to radially expand to a position for contacting and removing a target thrombus.

### CROSS-REFERENCE TO RELATED APPLICATIONS

This application claims priority to and the benefit of U.S. Provisional Patent Application No. 63/434,427 , filed Dec. 21, 2022 and titled “Thrombus Retriever Guidewire Device,” the entirety of which is incorporated herein by reference.

### BACKGROUND

### Technical Field

This disclosure relates generally to intravascular devices configured to engage, disrupt, capture, and/or remove thrombi, and methods of use thereof.

### Related Technology

A thrombus is a blood clot formed in situ within the vascular system. Thrombi may impede blood and/or portions may break away from the original site of formation when they embolize. Thrombi and emboli can cause a variety of health issues, including stroke, heart attack, deep vein thrombosis, pulmonary embolism, and other serious problems.

A standard thrombectomy procedure involves routing a catheter to the blockage and deploying a stent retriever to attempt to capture the clot. However, the incidence of complications and failures associated with such procedures remains relatively high. Accordingly, there is an ongoing need for improved devices capable of disrupting and/or removing thrombi.

### SUMMARY

Embodiments of the present disclosure relate to intravascular devices (such as guidewires) that include features to enable retrieval, disruption, and/or removal of a target thrombus. The term “thrombus,” as used herein, includes typical thrombi located at the position they were formed as well as thrombi that have broken loose from their original position and travelled to another location in the vasculature (i.e., emboli).

In one embodiment, a guidewire device includes a core having a proximal section and a distal section. A tube structure is coupled to the core such that the distal section extends into the tube structure. The tube structure may also be coupled to the core at the distal end of the core. The tube structure may be microfabricated to include a plurality of circumferentially extending “rings” joined by axially extending “beams” and/or may include other slots or fenestrations to tailor flexibility of the tube structure. The tube structure may include one or more sections defined by the number of beams disposed between each pair of successive rings, such as a one-beam section, two-beam section, and/or three-beam section. Such sections may also be referred to herein as including a one-beam cut pattern, two-beam cut pattern, and/or three-beam cut pattern, respectively.

In some embodiments, a wire mesh is disposed between sections of the tube structure. The terms wire mesh and braid are used interchangeably herein. A distal end of the wire mesh is connected to a first section of the tube structure and a proximal end of the wire mesh is connected to a second section of the tube structure. In some embodiments, the wire mesh can be connected to the first and second sections with, for example, an adhesive, soldering, and/or a mechanical connection. The first and second sections of the tube structure can include different cut patterns. For example, the first section can include a one-beam section and the second section can include a two-beam section.

The wire mesh allows the tube structure to contract along the longitudinal axis of the device when a proximal pulling force is applied to the core. Additionally, the wire mesh is configured to expand radially upon application of the proximal pulling force. The wire mesh is thus configured to radially expand to disrupt and/or facilitate the removal of thrombi within the patient.

In some embodiments, a radiopaque marker is disposed at or near the wire mesh, such as at or near the distal end of the wire mesh. In some embodiments, a radiopaque marker is additionally or alternatively disposed at or near the distal end of the device, such as at or near the distal end of the core and/or the tube structure.

The intravascular device can therefore include a core having a proximal section and a distal section, with a tube structure coupled to the distal section of the core, wherein the tube structure includes a first section and a second section proximal of the first section. A wire mesh is disposed in and extends across a gapped region of the tube structure, wherein the gapped region is disposed between the first section and the second section.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an embodiment showing certain structural features of an example intravascular device;

FIG. 2 illustrates an embodiment of an intravascular device comprising a wire mesh configured for selective radial expansion to enable disruption and/or removal of a target thrombus;

FIG. 3 illustrates another view of the intravascular device of FIG. 2 with the tube structure removed to better illustrate internal components; and

FIGS. 4A-4C illustrate an example method of using the intravascular device within a vessel to retrieve a target thrombus.

### DETAILED DESCRIPTION

Embodiments of the present disclosure relate to intravascular devices (such as guidewires or catheters) that include features to enable disruption and/or retrieval of a target thrombus.

In some embodiments, a wire mesh is disposed between sections of a tube structure (also referred to herein as simply “the tube”). For example, a distal end of the wire mesh may be connected to a first section of the tube structure and a proximal end of the wire mesh may be connected to a second section of the tube structure. The wire mesh can be connected to the sections with, for example, an adhesive, soldering, and/or through mechanical fixation.

The wire mesh allows the tube structure to contract along a longitudinal axis when a proximal pulling force is applied to the core to pull the first section of the tube structure closer to the second section of the tube structure. That is, the core can be connected to the first section of the tube structure but be free to longitudinally translate relative to the second section of the tube structure. When the core is “pulled,” the motion can thereby bring the first section of the tube structure closer to the second section of the tube structure, longitudinally contracting the tube structure. The wire mesh also expands radially upon application of the proximal pulling force and contraction of the tube structure. The user can thus move the wire mesh between a compact configuration and an expanded configuration to enable disruption and/or removal of thrombi.

In use, the device can be pushed through a thrombus until the meshed section passes distally beyond a target thrombus. The core wire can then be retracted to longitudinally contract the tube structure and radially expand the wire mesh. The device can then be retracted through the vasculature to bring the expanded wire mesh into contact with the thrombus. Further retraction of the device can dislodge and/or remove the thrombus. Additionally, or alternatively, the device can be rotated while in contact with the thrombus so that the expanded wire mesh agitates and breaks up the thrombus.

The device may be utilized in conjunction with an aspiration catheter. For example, the device can function as a guidewire for the aspiration catheter and the aspiration catheter can be positioned to retrieve the thrombus once it has been dislodged.

### Example Guidewire Structural Features

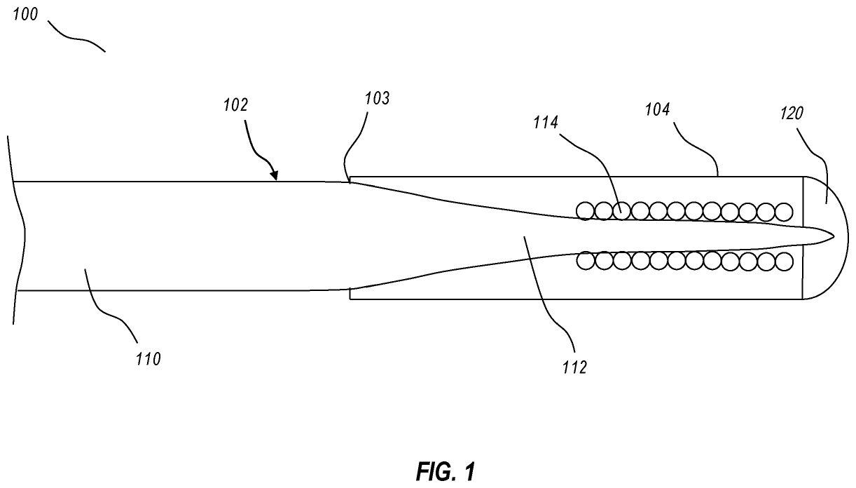

FIG. 1 illustrates a guidewire device 100 with a core 102 and a tube 104 encompassing a distal section of the core 102. In some embodiments, the core 102 includes one or more tapering sections so that the core 102 is able to fit within and extend into the tube 104. For example, the distal section of the core 102 may be ground so as to progressively taper to a smaller diameter at the distal end. In this example, the core 102 and the tube 104 have substantially similar outer diameters at the point where they adjoin one another. In other embodiments, the core 102 and the tube 104 can have different outer diameters at the point where they adjoin one another.

A medical grade adhesive may be used to couple the tube 104 to the core wire 102 at the distal end 120 of the device and to form an atraumatic covering. The tube 104 can be microfabricated to include a plurality of cuts that form circumferentially extending rings connected by axially extending beams. Examples of such cut patterns are described in U.S. Pat. No. 11,369,351 , which is incorporated herein by reference in its entirety. The tube 104 may additionally or alternatively include other slot or fenestration patterns.

The device 100 can have a length for effective delivery to a targeted anatomical area. For example, the device 100 can have a length ranging from 50 cm to 350 cm, or other lengths based on particular application needs. The device may have a diameter suitable for intravascular applications. Example sizes include, but are not limited to, standard guidewire sizes such as 0.010 inches, 0.014 inches, 0.024 inches, 0.035 inches, or larger sizes where appropriate. In some embodiments, the tube 104 has a length within a range of 3 cm to 100 cm.

The distal section 112 of the core 102 may taper to a diameter within a range of 0.001 to 0.050 inches, such as a diameter of 0.002 inches. In some embodiments, the distal section 112 of the core 102 tapers to a round cross-section. In other embodiments, the distal section 112 of the core 102 has a flat or rectangular cross-section. The distal section 112 may also have another cross-sectional shape, such as another polygon shape, an ovoid shape, an erratic shape, or combination of different cross-sectional shapes at different areas along its length.

Typically, a user will shape the distal end of the guidewire device 100 by manually bending, twisting, or otherwise manipulating the distal 1 cm to 3 cm (approximately) of the guidewire device 100 to a desired shape. This length is shown schematically as the distal “tip” 106 in FIG. 1.

As shown, the device 100 may also include a coil 114 positioned upon at least a portion of the distal section 112 of the core 102. The coil 114 is preferably formed from one or more radiopaque materials, such as platinum group, gold, silver, palladium, iridium, osmium, tantalum, tungsten, bismuth, dysprosium, gadolinium, and the like. Additionally, or alternatively, the coil 114 may be at least partially formed from a stainless steel or other material capable of effectively holding shaped after being bent or otherwise manipulated by a user. In some embodiments, the coil 114 has a length that substantially coincides with the length of the tube 104. In other embodiments, the coil 114 is shorter. Some embodiments may include two or more coils.

Although the illustrated embodiment shows a space between the coil 114 and the tube 104, in some embodiments, the coil 114 (or a set of multiple coils) is sized to fill and pack a greater proportion of the space between the distal section 112 and the tube 104. For example, the coil 114 (or a set of multiple coils) may be sized so as to abut both the core 102 and the inner surface of the tube 104. The coil 114 (or a set of multiple coils) may therefore function to pack the space between the core 102 and the tube 104 so as to align the curvature of the core 102 with the curvature of the tube 104.

For example, when a curvature is formed in the tube 104, the closely packed segments of the coil 114 (or a set of multiple coils) functions as a packing between the tube 104 and the core 102 to impart the same curvature to the core 102. In contrast, a guidewire device omitting such features would, when curved at the tube, not follow the same curve as the tube but would extend until abutting against the inner surface of the tube before being forced to curve. Aligning the curvature of the core 102 and the tube 104 can beneficially minimize the buildup of mismatched forces within the core 102 which, when released, can detrimentally cause the distal end of the device to “whip.” Such whipping can disrupt intended guidewire navigation or even damage sensitive anatomy.

### Wire Mesh/Braid Components

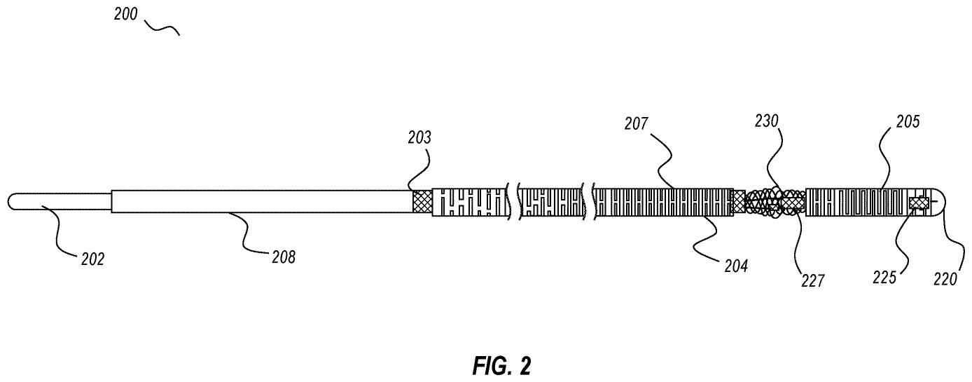

FIG. 2 illustrates an embodiment of a guidewire device 200 including a wire mesh feature configured to enable retrieval of thrombi. The general disclosure related to FIG. 1 is also applicable to the guidewire device 200, and any of the components or features described above are also applicable to the guidewire device 200.

As illustrated, the tube structure 204 includes a first section 205 and a second section 207, with a gapped region disposed between the first section 205 and second section 207. That is, there is an absence of tube material or structure within the gapped region. The wire mesh 230 is disposed within and extends across the gapped region, bridging the first and second sections 205, 207.

The illustrated guidewire device 200 includes a second tube structure, referred to herein as actuation tube 208 (e.g., a hypotube) that is proximal to the tube structure 204. The tube structure 204 attaches to the actuation tube 208 at attachment point 203 such as via an adhesive, soldering, and/or mechanical fixation. The actuation tube 208 can extend proximally to, for example, a handle (not shown) along with the core 202. The core 202 can extend farther proximally than the actuation tube 208 such that the proximal end of the core 202 can be selectively translated/pulled relative to the actuation tube 208.

The core 202 is attached to the first section 205 of the tube structure 204 at the distal end 220 but is free to translate relative to the second section 207. Accordingly, retraction of the core 202 brings the first section 205 closer to the second section 207. This reduces the size of the gapped region and causes the wire mesh 230 to expand radially. The wire mesh 230 is constructed to allow longitudinal contraction and radial expansion in response to translation of the core 202 relative to the second section 207 and actuation tube 208.

A medical grade adhesive may be used to couple the tube 204 to the core 202 at the distal end 220. In some embodiments, the distal end 220 forms an atraumatic covering. In other embodiments, the distal end 220 incorporates a scraping edge and/or drill tip to facilitate protrusion of the device into and optionally through a target thrombus.

In some embodiments, the first section 205 and the second section 207 include different cut patterns. In some embodiments, the second section 207 includes more than one cut pattern. For example, the second section 207 can include a three-beam section and a two-beam section. In some embodiments, the first section 205 includes more than one cut pattern. For example, the first section 205 can include a two-beam section and a one-beam section. Beneficially, including a two-beam section adjacent the wire mesh 230 (e.g., on both sides) provides sufficient structure or surface area to which an adhesive can adhere, thereby contributing to a stronger connection between the wire mesh 230 and the adjacent sections 205, 207 of the tube structure 204.

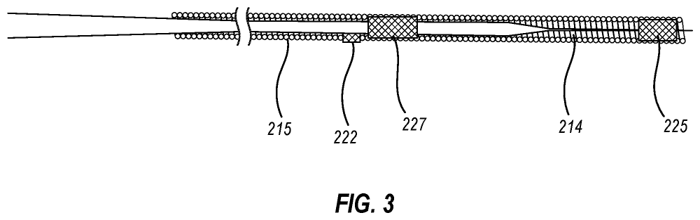

FIG. 3 illustrates a cross-sectional view of the guidewire device 200 with tube structure 204 and actuation tube 208 removed to better illustrate certain internal components. As shown, device 200 includes a distal coil 214 and a proximal coil 215 positioned around core 202. The coils 214 and 215 may be independently formed from, for example, stainless steel or a radiopaque material. In the illustrated embodiment, the coil 214 is disposed at or near the distal end of the device and extends a distance proximally toward the radiopaque marker 227. The coil 215 is disposed proximal of coil 214. An adhesive 222 may be used to attach the coil 215 to the core 202. In the illustrated embodiment, the wire mesh 230 is positioned to span both coil 214 and coil 215. That is, the wire mesh 230 is coincident with a distal portion of coil 215 and with a proximal portion of coil 214.

As illustrated, a distal most end of the core 202 extends past the coil 214 to form part of the distal end 220. In some embodiments, as discussed above with respect to FIG. 1, the coil 214 and/or the coil 215 is/are sized to fill and pack the space between the core 202 and the tube structure 204. For example, the coil 214 and/or the coil 215 may be sized so as to abut both the core 202 and the inner surface of the tube structure 204 along at least a portion of the length of the tube structure 204.

For example, the coil 214 and/or the coil 215 may be sized so as to abut both the core 202 and the inner surface of the tube structure 204 along at least 20% of the length of the tube structure 204, or along at least 30% of the length of the tube structure 204, or along at least 40% of the length of the tube structure 204, or along at least 50% of the length of the tube structure 204, or along at least 60% of the length of the tube structure 204, or along at least 70% of the length of the tube structure 204, or along at least 80% of the length of the tube structure 204, or along at least 90% of the length of the tube structure 204.

### Example Method of Use

In use, the guidewire device 200 can be routed through a patient's vasculature toward a target thrombus. The distal end 220 and the first section 205 of the tube structure 204 can be advanced through the thrombus. A portion of the wire mesh 230 may also be advanced through the thrombus. Placement and/or advancement of the distal end 220 can be monitored and ascertained through a radiopaque marker 225 disposed near the distal end 220. In some embodiments, a second radiopaque marker 227 is also disposed coincident with the wire mesh 230 (e.g., coincident with a distal portion of the wire mesh 230). The second radiopaque marker 227 beneficially enables the operator to ascertain when the wire mesh 230 is aligned with or has sufficiently passed through the target thrombus.

After the device 200 is appropriately positioned with respect to the thrombus, a proximal pulling force can be applied to the core 202. Application of the proximal pulling force brings the first section 205 and the second section 207 closer together, causing the wire mesh 230 to expand radially. The guidewire device 200 can then be retracted through the patient's vasculature, contacting the thrombus and enabling dislodging and/or evacuation of the thrombus (e.g., via an aspiration catheter).

In some embodiments, an aspiration catheter is also navigated to the thrombus (e.g., using the intravascular device as a guidewire) to aid in the removal of the thrombus from the patient.

In some embodiments, once the wire mesh 230 has been opened/expanded, the intravascular device 200 can be rotated back and forth to agitate and break up the thrombus, for example as suction is applied by an aspiration catheter to retrieve the broken thrombi. In some embodiments, a handle attached to a proximal end of the intravascular device 200 can control the expansion of the wire mesh 230 within the vasculature of the patient by controlling relative movement of the core 202 and the actuation tube 208, for example.

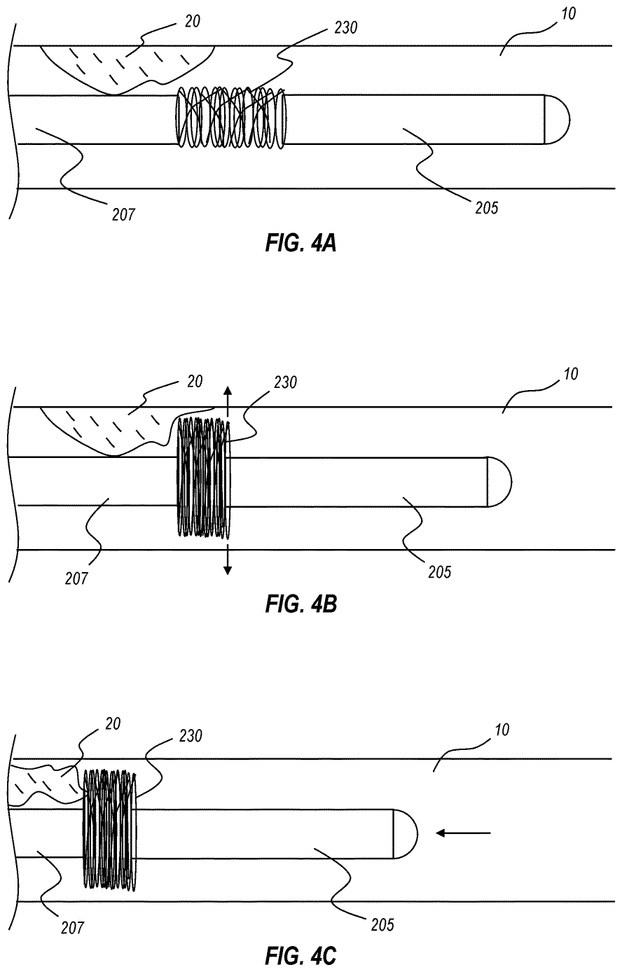

An example method of use is shown in FIGS. 4A-4C. FIG. 4A shows the device routed through a vessel 10 until the first section 205 is distally past a target thrombus 20 and the wire mesh 230 is aligned with or distally beyond the thrombus 20. As shown in FIG. 4B, the device may be actuated to bring the first section 205 closer to the second section 207 and thereby cause radial expansion of the wire mesh 230. In the expanded state, the wire mesh 230 is able to engage with the thrombus 20. The device can be rotated and/or longitudinally moved to help dislodge and/or break up the thrombus 20. As shown in FIG. 4C, retraction of the device while the wire mesh 230 is in the expanded state enables retrieval/removal of the thrombus 20 (e.g., toward an aspiration catheter).

### Example Embodiments

The following clauses represent a non-exhaustive list of example embodiments:
Clause 1. An intravascular device comprising: a core having a proximal section and a distal section; a tube structure coupled to the distal section of the core, wherein the tube structure comprises a first section and a second section, the second section being disposed proximal of the first section; and a wire mesh disposed in a gapped region of the tube structure, wherein the gapped region is disposed between the first section and the second section, wherein the core is coupled to the first section of the tube structure and is free to longitudinally translate relative to the second section of the tube structure such that when the core is moved proximally relative to the second section of the tube structure, the gapped region is collapsed, and the wire mesh is radially expanded.
Clause 2. The intravascular device of clause 1, further comprising a radiopaque marker longitudinally coincident with the wire mesh.
Clause 3. The intravascular device of any preceding clause, wherein the tube structure is coupled to the core at only the distal section of the core.
Clause 4. The intravascular device of any preceding clause, wherein the first section and/or second section of the tube structure includes a cut pattern comprising a plurality of circumferentially extending rings connected by axially extending beams.
Clause 5. The intravascular device of any preceding clause, wherein a distal end of the wire mesh is connected to the first section of the tube structure and a proximal end of the wire mesh is connected to the second section of the tube structure, thereby bridging the gapped region between the first and second sections.
Clause 6. The intravascular device of clause 5, wherein the wire mesh is connected to the first and second sections by an adhesive.
Clause 7. The intravascular device of any preceding clause, further comprising one or more coils disposed within the tube structure so as to be positioned between an outer surface of the distal section of the core and an inner surface of the tube structure.
Clause 8. The intravascular device of clause 7, wherein the one or more coils comprise a distal coil and a separate proximal coil.
Clause 9. The intravascular device of clause 8, wherein the wire mesh is longitudinally coincident with at least a portion of the proximal coil and at least a portion of the distal coil.
Clause 10. The intravascular device of any preceding clause, wherein the first section comprises a first cut pattern and a different, second cut pattern.
Clause 11. The intravascular device of any preceding clause, wherein the second section comprises a first cut pattern and a different, second cut pattern.
Clause 12. The intravascular device of any preceding clause, further comprising a radiopaque marker disposed at or near a distal tip of the device.
Clause 13. The intravascular device of any preceding clause, further comprising an actuation tube attached to a proximal end of the tube structure and extending proximally therefrom.
Clause 14. The intravascular device of clause 13, wherein the actuation tube is a hypotube.
Clause 15. The intravascular device of clause 13 or clause 14, wherein the actuation tube omits a cut pattern and omits rings and beams.
Clause 16. The intravascular device of any one of clauses 13-15, wherein the core translates with the first section and is longitudinally translatable relative to the second section and the actuation tube.
Clause 17. An intravascular device comprising: a core having a proximal section and a distal section; a tube structure coupled to the distal section of the core, wherein the tube structure comprises a first section and a second section, the second section being disposed proximal of the first section; a wire mesh disposed in a gapped region of the tube structure, wherein the gapped region is disposed between the first section and the second section; a first radiopaque marker disposed at or near a distal tip of the device and longitudinally coincident with the first section of the tube structure; a second radiopaque marker longitudinally coincident with the wire mesh; and an actuation tube attached to a proximal end of the tube structure and extending proximally therefrom, wherein the core is coupled to the first section of the tube structure and is free to longitudinally translate relative to the second section of the tube structure and relative to the actuation tube such that when the core is moved proximally relative to the second section of the tube structure and relative to the actuation tube, the gapped region is collapsed, and the wire mesh is radially expanded.
Clause 18. The intravascular device of clause 17, further comprising one or more coils disposed within the tube structure so as to be positioned between an outer surface of the distal section of the core and an inner surface of the tube structure, wherein the one or more coils comprise a distal coil and a separate proximal coil, and wherein the wire mesh is longitudinally coincident with the proximal coil and the distal coil.
Clause 19. A method of removing a thrombus from a patient, the method comprising: navigating an intravascular device through vasculature of the patient toward the thrombus, wherein the intravascular device is an intravascular device as in any one of clauses 1-18; expanding a wire mesh of the intravascular device; retracting the intravascular device and contacting the expanded wire mesh to the thrombus; and dislodging at least a portion of the thrombus.
Clause 20. The method of clause 19, further comprising applying a proximal pulling force to a core of the intravascular device to contract the tube structure and expand the wire mesh.

### Additional Terms & Definitions

As used herein, the term “longitudinally coincident” (sometimes shortened to simply “coincident”) means two components that are both disposed at a given longitudinal position of the device and may, for example, overlap at least partially. For example, if a first component and a second component are longitudinally coincident, at least a portion of the first component overlaps with at least a portion of the second component at some location of the device.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device comprising:
a core having a proximal section and a distal section;
a tube structure coupled to the distal section of the core, wherein the tube structure comprises a first section and a second section, the second section being disposed proximal of the first section; and
a wire mesh disposed in a gapped region of the tube structure, wherein the gapped region is disposed between the first section and the second section,
wherein the core is coupled to the first section of the tube structure and is free to longitudinally translate relative to the second section of the tube structure such that when the core is moved proximally relative to the second section of the tube structure, the gapped region is collapsed, and the wire mesh is radially expanded.

2. The intravascular device of claim 1, further comprising a radiopaque marker longitudinally coincident with the wire mesh.

3. The intravascular device of claim 1, wherein the tube structure is coupled to the core at only the distal section of the core.

4. The intravascular device of claim 1, wherein the first section and/or second section of the tube structure includes a cut pattern comprising a plurality of circumferentially extending rings connected by axially extending beams.

5. The intravascular device of claim 1, wherein a distal end of the wire mesh is connected to the first section of the tube structure and a proximal end of the wire mesh is connected to the second section of the tube structure, thereby bridging the gapped region between the first and second sections.

6. The intravascular device of claim 5, wherein the wire mesh is connected to the first and second sections by an adhesive.

7. The intravascular device of claim 1, further comprising one or more coils disposed within the tube structure so as to be positioned between an outer surface of the distal section of the core and an inner surface of the tube structure.

8. The intravascular device of claim 7, wherein the one or more coils comprise a distal coil and a separate proximal coil.

9. The intravascular device of claim 8, wherein the wire mesh is longitudinally coincident with at least a portion of the proximal coil and at least a portion of the distal coil.

10. The intravascular device of claim 1, wherein the first section comprises a first cut pattern and a different, second cut pattern.

11. The intravascular device of claim 1, wherein the second section comprises a first cut pattern and a different, second cut pattern.

12. The intravascular device of any claim 1, further comprising a radiopaque marker disposed at or near a distal tip of the device.

13. The intravascular device of claim 1, further comprising an actuation tube attached to a proximal end of the tube structure and extending proximally therefrom.

14. The intravascular device of claim 13, wherein the actuation tube is a hypotube.

15. The intravascular device of claim 13, wherein the actuation tube omits a cut pattern and omits rings and beams.

16. The intravascular device of claim 13, wherein the core translates with the first section and is longitudinally translatable relative to the second section and the actuation tube.

17. An intravascular device comprising:
a core having a proximal section and a distal section;
a tube structure coupled to the distal section of the core, wherein the tube structure comprises a first section and a second section, the second section being disposed proximal of the first section;
a wire mesh disposed in a gapped region of the tube structure, wherein the gapped region is disposed between the first section and the second section;
a first radiopaque marker disposed at or near a distal tip of the device and longitudinally coincident with the first section of the tube structure;
a second radiopaque marker longitudinally coincident with the wire mesh; and
an actuation tube attached to a proximal end of the tube structure and extending proximally therefrom,
wherein the core is coupled to the first section of the tube structure and is free to longitudinally translate relative to the second section of the tube structure and relative to the actuation tube such that when the core is moved proximally relative to the second section of the tube structure and relative to the actuation tube, the gapped region is collapsed, and the wire mesh is radially expanded.

18. The intravascular device of claim 17, further comprising one or more coils disposed within the tube structure so as to be positioned between an outer surface of the distal section of the core and an inner surface of the tube structure, wherein the one or more coils comprise a distal coil and a separate proximal coil, and wherein the wire mesh is longitudinally coincident with the proximal coil and the distal coil.

19. A method of removing a thrombus from a patient, the method comprising:
navigating an intravascular device through vasculature of the patient toward the thrombus, wherein the intravascular device is an intravascular device as in any one of claims 1-18;
expanding a wire mesh of the intravascular device;
retracting the intravascular device and contacting the expanded wire mesh to the thrombus; and
dislodging at least a portion of the thrombus.

20. The method of claim 19, further comprising applying a proximal pulling force to a core of the intravascular device to contract the tube structure and expand the wire mesh.
